(19) United States
(12) Patent Application Publication
(10) Pub. No.: US 2022/0196591 A1
(43) Pub. Date: Jun. 23, 2022

(54) Wearable Sensor and Perspiration Analisys Device

(71) Applicant: Nippon Telegraph and Telephone Corporation, Tokyo (JP)

(72) Inventors: Yuki Hashimoto, Tokyo (JP); Kei Kuwabara, Tokyo (JP)

(21) Appl. No.: 17/598,765

(22) PCT Filed: May 8, 2019
(86) PCT No.: PCT/JP2019/018364

§ 371 Date: Sep. 27, 2021

(51) Int. Cl. G01N 27/333; G01N 27/327; G01N 27/414; A61B 5/1486; A61B 5/145; A61B 5/00

(52) U.S. Cl. CPC G01N 27/333; G01N 27/3271; G01N 27/4145; A61B 5/1486; A61B 5/0002; A61B 5/6801; A61B 5/14546; A61B 5/14532; A61B 5/14517

(57) ABSTRACT

A wearable sensor (1) includes a base member (10) including a flow channel (11), a sensor element (12) provided in the flow channel (11) and configured to detect a signal related to an electrical characteristic of a liquid in the flow channel (11), and a porous body (15) having hydrophilicity and disposed on an inner wall of the flow channel (11) in a portion farther from a position of the sensor element (12) when viewed from a first opening of the flow channel (11), and on a surface of the base member on a side where a second end portion on a side opposite to the first opening opens.

### CROSS-REFERENCE TO RELATED APPLICATION

This application is a national phase entry of PCT Application No. PCT/JP2019/018364, filed on May 8, 2019, which application is hereby incorporated herein by reference.

### TECHNICAL FIELD

The present invention relates to a wearable sensor and a perspiration analysis device for analyzing a component in perspiration of a person.

### BACKGROUND

In a human body, there are tissues, such as muscles and nerves, that perform electrical activities, and in order to keep these tissues operating normally, a mechanism exists that keeps a concentration of electrolytes in the body constant mainly by the workings of the autonomic nervous system and the endocrine system. For example, when a large amount of electrolytes in the body is lost as a result of perspiration due to long-term exposure to a hot environment, excessive exercise, or the like and the electrolyte concentration in the body deviates from normal values, various symptoms represented by heat stroke occur.

In recent years, against this backdrop, research has been conducted to understand electrolyte abnormalities in the human body by monitoring the electrolyte concentration in perspiration. For example, Non Patent Literature 1 proposes a wearable device for monitoring the electrolyte ion concentration in perspiration and, from measurement results by this device, it has become clear that the electrolyte ion concentration is useful as a biomarker for dehydration.

Considering that the electrolyte concentration in perspiration is continuously measured for a long time with the device attached to the human body, for example, when strenuous exercise is performed for a certain period of time, and then a break is subsequently taken, and exercise is resumed, that is, when a person perspires for a certain period of time, subsequently stops perspiring, and then perspires again, there is a problem in that the electrolyte ions from the previous perspiration dry, the dried electrolyte ions adhere to the sensor element as salt, and the salt redissolves when perspiration resumes, which affects the measurement.

### CITATION LIST

### Non Patent Literature

Non Patent Literature 1: Wei Gao, et al., “Fully Integrated Wearable Arrays for Multiplexed In Situ Perspiration Analysis,” Nature, Vol. 529, 509-526, 2016

### SUMMARY

### Technical Problem

In order to solve the problems described above, an object of embodiments of the present invention is to provide a wearable sensor and a perspiration analysis device capable of reducing the influence that perspiration drying has on component analysis and achieving long-term analysis of a component in perspiration.

### Means for Solving the Problem

A wearable sensor according to embodiments of the present invention includes a base member including a through-hole, a first sensor element provided in the through-hole and configured to detect a signal related to an electrical characteristic of a liquid in the through-hole, and a porous body having hydrophilicity and disposed on an inner wall of the through-hole in a portion farther from a position of the first sensor element when viewed from a first opening of the through-hole, and on a surface of the base member on a side where a second end portion on a side opposite to the first opening opens.

Further, in one configuration example of the wearable sensor according to embodiments of the present invention, when the base member is attached to a body of the wearer with the base member facing skin of the wearer, a first end portion of the through-hole opens on a side of the base member that faces the skin of the wearer, the first sensor element is disposed in the through-hole and detects an electrical signal derived from an analysis target component contained in perspiration that has flowed into the through-hole from the first opening, and at least the inner wall of the through-hole has hydrophilicity.

Further, in one configuration example of the wearable sensor according to embodiments of the present invention, the wearable sensor further includes a water-repellent member provided on a surface of the base member on a side where the first end portion opens.

Further, a perspiration analysis device according to embodiments of the present invention includes the wearable sensor and a component concentration calculation unit configured to calculate a concentration of the analysis target component from an electrical signal detected by the first sensor element of the wearable sensor.

Further, in one configuration example of the perspiration analysis device according to embodiments of the present invention, the component concentration calculation unit determines that acquisition of the concentration of the analysis target component is completed when a value of the concentration of the analysis target component is stable.

Further, in one configuration example of the perspiration analysis device according to embodiments of the present invention, the wearable sensor further includes a second sensor element for perspiration detection disposed in the through-hole at a position adjacent to the first sensor element, and the component concentration calculation unit determines that the acquisition of the concentration of the analysis target component is completed when perspiration secreted from the skin of the wearer is detected by the second sensor element.

Further, in one configuration example of the perspiration analysis device according to embodiments of the present invention, the perspiration analysis device further includes a communication unit configured to transmit, to an external device, the value of the concentration of the analysis target component calculated by the component concentration calculation unit.

### Effects of Embodiments of the Invention

According to embodiments of the present invention, it is possible to adjust the surface tension of a solution (perspiration) so that the solution reaches a position of the porous body by capillary action due to the size of the through-hole, the positions of the first sensor element and the porous body, and the hydrophilicity of the inner wall of the through-hole. Then, according to embodiments of the present invention, the solution moves through a large number of pores of the porous body toward the opening on a side opposite to the skin by capillary action, and evaporates while moving in the porous body on the surface of the base member on a side opposite to the skin. As a result, according to embodiments of the present invention, in wearable-form component analysis, it is possible to reduce adhesion of salt to the surface of the first sensor element and achieve long-term analysis of a component in the solution.

### BRIEF DESCRIPTION OF DRAWINGS

FIG. 1 is a block diagram illustrating a configuration of a perspiration analysis device according to an embodiment of the present invention.

FIG. 2 is a functional block diagram of a micro control unit (MCU) of the perspiration analysis device according to the embodiment of the present invention.

FIG. 3 is a cross-sectional view illustrating a configuration of a wearable sensor of the perspiration analysis device according to the embodiment of the present invention.

FIG. 4 is a flowchart for describing an operation of the perspiration analysis device according to the embodiment of the present invention.

FIG. 5 is a cross-sectional view illustrating how perspiration of a wearer is made to rise in a flow channel in the embodiment of the present invention.

FIG. 6 is a cross-sectional view illustrating how the perspiration of the wearer is made to rise in the flow channel and thus reach a position of a sensor element in the embodiment of the present invention.

FIG. 7 is a cross-sectional view illustrating how the perspiration of the wearer is made to rise in the flow channel and thus reach a position of a porous body in the embodiment of the present invention.

FIG. 8 is a cross-sectional view illustrating how the perspiration of the wearer moves to a side opposite to the skin in the embodiment of the present invention.

FIG. 9 is a block diagram illustrating a configuration example of a computer that realizes the perspiration analysis device according to an embodiment of the present invention.

### DESCRIPTION OF EMBODIMENTS

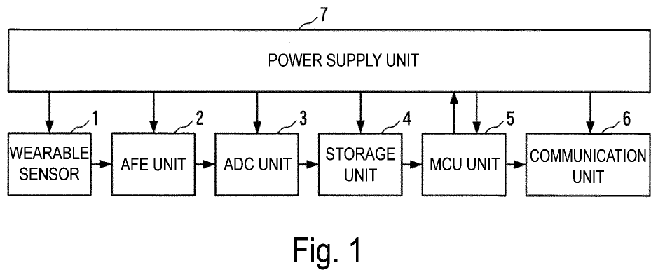

Embodiments of the present invention will be described below with reference to the drawings. FIG. 1 is a block diagram illustrating a configuration of a perspiration analysis device according to an embodiment of the present invention. The perspiration analysis device includes a wearable sensor 1, an analog front end (AFE) unit 2, an analog to digital converter (ADC) unit 3, a storage unit 4, a micro control unit (MCU) 5, a communication unit 6, and a power supply unit 7.

The wearable sensor 1 detects an electrical signal derived from an analysis target component in perspiration secreted from the skin of a wearer.

The AFE unit 2 is a circuit that includes an analog front end and amplifies a faint electrical signal detected by the wearable sensor 1.

The ADC unit 3 includes an analog-digital converter, and is a circuit that converts an analog signal amplified by the AFE unit 2 into digital data at a predetermined sampling frequency.

The storage unit 4 stores digital data output by the ADC unit 3. The storage unit 4 is realized by a non-volatile memory typified by a flash memory, a volatile memory such as a dynamic random access memory (DRAM), or the like.

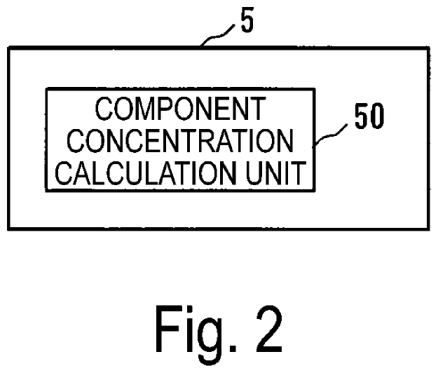

The MCU 5 is a circuit responsible for signal processing that calculates the concentration of the analysis target component from the digital data stored in the storage unit 4. FIG. 2 is a functional block diagram of the MCU 5. The MCU 5 is a circuit that functions as a component concentration calculation unit 50.

The communication unit 6 includes a circuit that wirelessly or wiredly transmits an analysis result obtained by the MCU 5 to an external device (not illustrated) such as a smartphone. Examples of standards for wireless communication include Bluetooth (trade name) Low Energy (BLE) and the like. Further, examples of standards for wired communication include Ethernet (trade name) and the like.

The power supply unit 7 is a circuit responsible for supplying power to the perspiration analysis device.

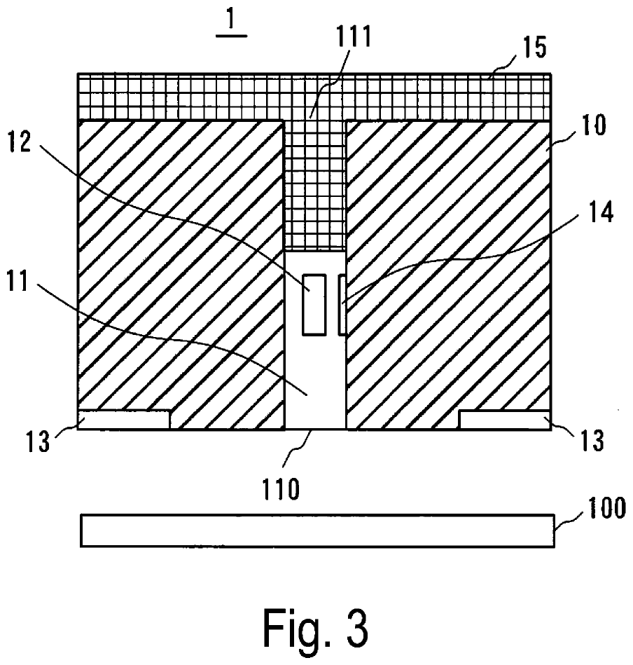

FIG. 3 is a cross-sectional view illustrating a configuration of the wearable sensor 1. The wearable sensor 1 includes a base member 10 mounted on the body of the wearer of the wearable sensor 1 so as to face skin 100 of the wearer, a flow channel 11 (through-hole) having a hole shape, a sensor element 12 (first sensor element), a water-repellent member 13, a water detection sensor element 14 (second sensor element), and a porous body 15 having hydrophilicity. The flow channel 11 (through-hole) is a flow channel having a hole shape and formed through the base member 10 so that one opening 110 faces the skin of the wearer. The sensor element 12 (first sensor element) is disposed in the flow channel 11 and is a sensor element that detects an electrical signal derived from the analysis target component in the perspiration secreted from the skin 100 of the wearer and made to flow into the flow channel 11. The water-repellent member 13 is provided on a surface of the base member 10 on the skin 100 side. The water detection sensor element 14 (second sensor element) is a water detection sensor element disposed in the flow channel 11 at a position adjacent to the sensor element 12. The porous body 15 is a porous body having hydrophilicity and disposed in the flow channel 11 at a position further from the skin 100 than a position of the sensor element 12 and on a surface (top surface in FIG. 3) of the base member 10 on a side opposite to the skin 100.

Examples of the base member 10 include a base member made of a glass material having hydrophilicity or a resin material having hydrophilicity. Further, the base member 10 may be a base member subjected to a surface treatment that imparts hydrophilicity to a surface of a water-repellent material and an inner wall of the flow channel 11. The diameter of the flow channel 11 formed in the base member 10 is, for example, about several mm.

When a hydrophilic material is used for the base member 10, it is only required that the water-repellent member 13 be formed by applying a water-repellent surface treatment to the surface (lower surface in FIG. 3) of the base member 10 on the skin 100 side. When a water-repellent material is used for the base member 10, this surface on the skin 100 side can be made into the water-repellent member 13 by applying a hydrophilic surface treatment to the surface of the base member 10 (top surface in FIG. 3) on the side opposite to the skin 100 and to the inner wall of the flow channel 11, leaving only the surface of the base member 10 on the skin 100 side as the water-repellent material as is. In the example in FIG. 3, the water-repellent member 13 is disposed at a position away from the opening 110, but the water-repellent member 13 may be disposed near the opening 110.

Examples of the sensor element 12 include an ion selective electrode used in Non Patent Literature 1, an enzyme electrode, and an ion-sensitive field effect transistor.

The sensor element 12 is, for example, formed on an inner wall surface of the flow channel 11. Note that, in order to analyze a plurality of components in the perspiration, a plurality of the sensor elements 12 having selectivity of the target component may be provided.

Examples of the porous body 15 having hydrophilicity include porous bodies derived from hydrophilic materials such as nylon and cellulose.

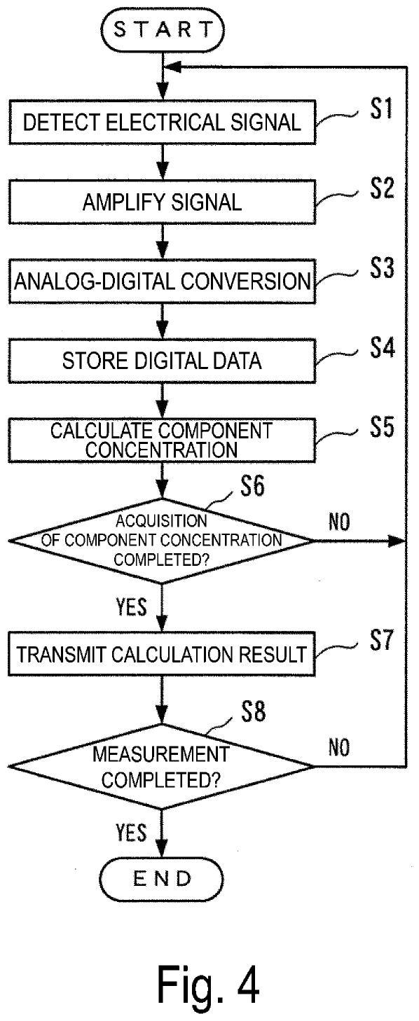

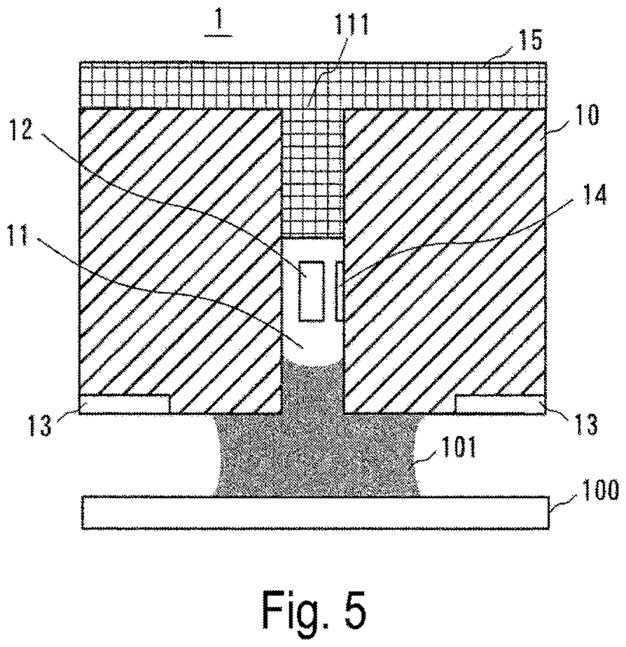

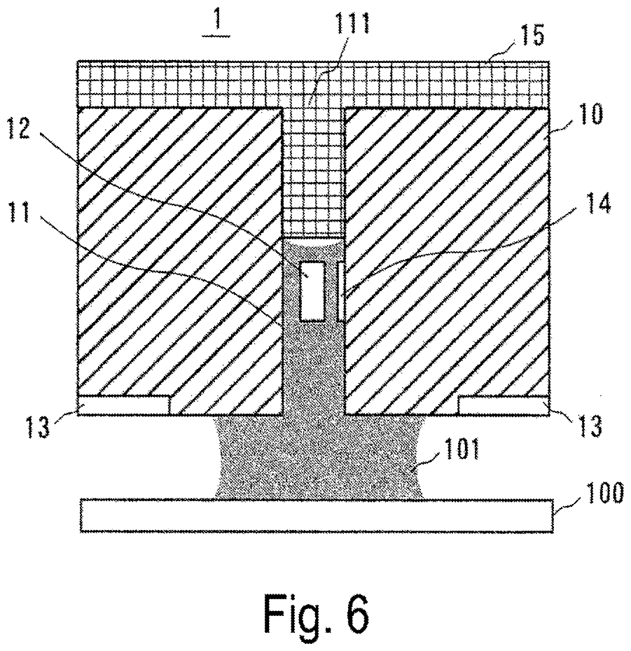

FIG. 4 is a flowchart for describing an operation of the perspiration analysis device according to this embodiment. At the start of measurement, a liquid droplet of perspiration is formed between the skin 100 and the flow channel 11 of the wearer by a hydrophilic/hydrophobic pattern of an inlet portion of the flow channel 11 (pattern in which the inner wall of the flow channel 11 is hydrophilic and the water-repellent member 13 in the periphery is hydrophobic). Then, through capillary action, perspiration 101 is introduced into the flow channel 11 (FIG. 5). Furthermore, with an increase in perspiration amount, the perspiration 101 moves inside the flow channel 11 and reaches the position of the sensor element 12 in the flow channel 11 (FIG. 6).

It is only required that the diameter of the flow channel 11, the length of the flow channel 11, the positions of the sensor element 12 and the porous body 15 within the flow channel 11, and the hydrophilicity (wettability) of the inner wall of the flow channel 11 be set so that the perspiration 101 reaches the position of the porous body 15 by capillary action.

The sensor element 12 detects an electrical signal derived from the analysis target component in the perspiration 101 (FIG. 4, step S1).

The AFE unit 2 amplifies a faint electrical signal detected by the sensor element 12 (FIG. 4, step S2).

The ADC unit 3 converts the analog signal amplified by the AFE unit 2 into digital data (FIG. 4, step S3). The digital data output from the ADC unit 3 is stored in the storage unit 4 (FIG. 4, step S4).

The component concentration calculation unit 50 calculates the concentration of the analysis target component from the digital data stored in the storage unit 4 (FIG. 4, step S5). Examples of the component concentration in the perspiration 101 include lactic acid concentration, glucose concentration, sodium ion concentration, and potassium ion concentration. Note that, as is clear from Non Patent Literature 1, the technique for calculating the component concentration is well known, and thus detailed descriptions thereof will be omitted.

Next, the component concentration calculation unit 50 determines, for example, that the acquisition of the component concentration is completed when the water detection sensor element 14 provided in the flow channel 11 at a position adjacent to the sensor element 12 detects that the perspiration 101 has reached the position of the sensor element 12 (YES in FIG. 4, step S6). Alternatively, the component concentration calculation unit 50 may determine that the value of the component concentration is stable and that the acquisition of the component concentration is completed when an amount of change per unit time of the calculated value of the component concentration is less than or equal to a predetermined threshold value.

When the acquisition of the component concentration is completed, the communication unit 6 transmits the value of the component concentration calculated by the component concentration calculation unit 50 to an external device (not illustrated) such as a smartphone (FIG. 4, step S7).

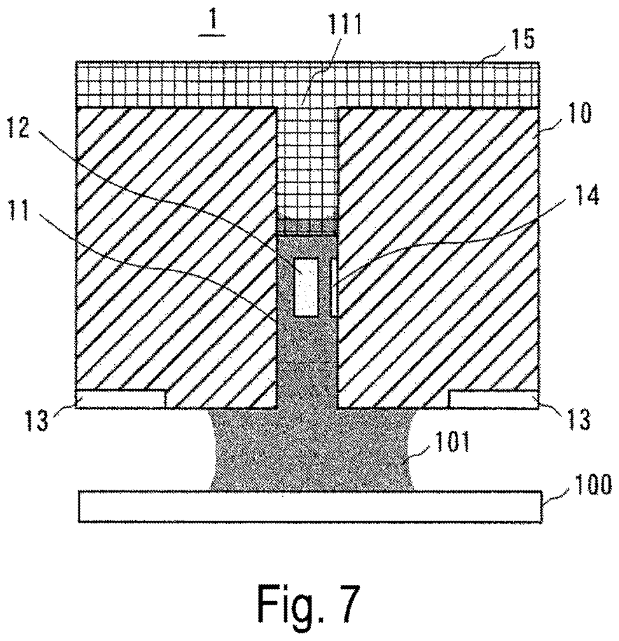

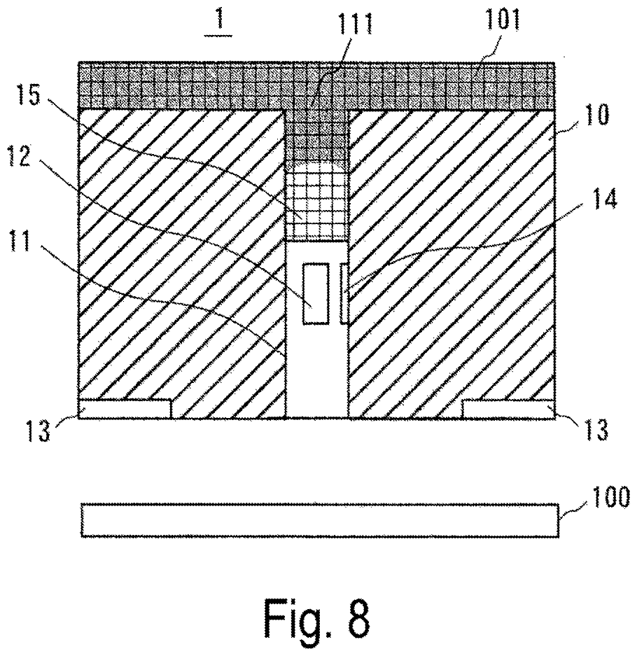

Furthermore, when the amount of perspiration increases, the perspiration 101 moves inside the flow channel 11 and reaches the position of the porous body 15 in the flow channel 11 (FIG. 7). The perspiration 101 that reaches the porous body 15 moves, by capillary action, through a plurality of the holes of the porous body 15 in the flow channel 11 toward the opening 111 on the side opposite to the skin 100 and evaporates while further moving inside the porous body 15 on the surface of the base member 10 on the side opposite to the skin 100 (FIG. 8). It is only required that the size of each hole of the porous body 15 and the hydrophilicity (wettability) of the porous body 15 be set so that the perspiration 101 diffuses to a region on the surface of the base member 10 on the side opposite to the skin 100 by capillary action. Thus, the perspiration 101 can be removed from the wearable sensor 1.

The perspiration analysis device repeatedly performs the processes of steps S1 to S7 until, for example, there is an instruction for measurement completion from the wearer (YES in FIG. 4, step S8).

As described above, according to this embodiment, in perspiration component analysis by a wearable form, it is possible to reduce adhesion of salt to the surface of the sensor element 12 and achieve long-term analysis of a component in the perspiration. Salt derived from dried electrolyte ions may adhere to the porous body 15 on the surface of the base member 10 opposite to the skin 100, but is in a position away from the skin 100 and the sensor element 12, making it unlikely that the salt adhered to the porous body 15 on the surface of the base member 10 will dissolve when perspiration resumes and reach the sensor element 12.

Further, in this embodiment, as long as the volume of the liquid droplets of the perspiration 101, which occurs between the skin 100 and the flow channel 11 of the wearer, and the surface area of the wearable sensor 1 in the region that comes into contact with the droplets can be estimated, a perspiration rate and a cumulative perspiration volume per unit area of the wearer can be calculated.

That is, the component concentration calculation unit 50 can calculate the cumulative perspiration amount of the wearer in a total elapsed time from completion of acquisition of the component concentration to completion of acquisition of the next component concentration by adding the known volume described above each time acquisition of the component concentration is completed.

Further, the component concentration calculation unit 50 can calculate the perspiration rate per unit area of the wearer by dividing the known volume described above by the elapsed time from completion of acquisition of the immediately preceding component concentration to completion of acquisition of the most recent component concentration and by the surface area described above, each time acquisition of the component concentration is completed.

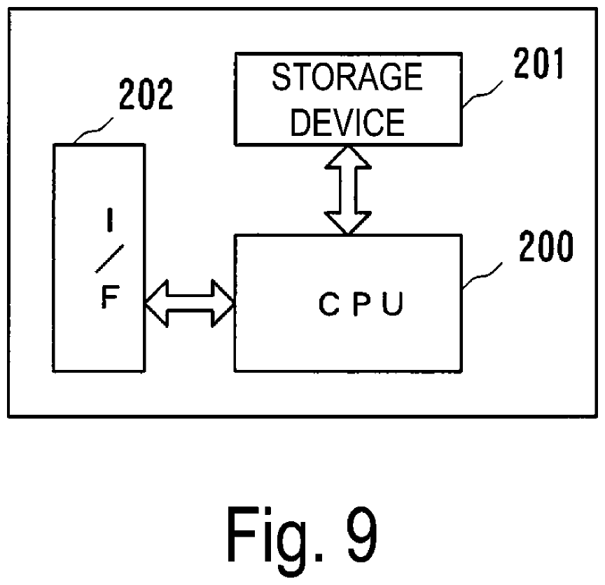

The storage unit 4 and the MCU 5 described in this embodiment can be realized by a computer including a central processing unit (CPU), a storage device, and an interface, and programs for controlling these hardware resources. A configuration example of this computer is illustrated in FIG. 9. The computer includes a CPU 200, a storage device 201, and an interface device (hereinafter abbreviated as I/F) 202. The ADC unit 3, the communication unit 6, the power supply unit 7, and the like are connected to the I/F 202. In such a computer, a program for realizing the perspiration analysis method of embodiments of the present invention is stored in the storage device 201.

The CPU 200 executes the processes described in this embodiment in accordance with the program stored in the storage device 201.

### INDUSTRIAL APPLICABILITY

Embodiments of the present invention can be applied to techniques for analyzing a component in a perspiration of a person.

### REFERENCE SIGNS LIST

1 Wearable sensor
2 AFE unit
3 ADC unit
4 Storage unit
5 MCU unit
6 Communication unit
7 Power supply unit
10 Base member
11 Flow channel
12 Sensor element
13 Water-repellent member
14 Water detection sensor element
15 Porous body
50 Component concentration calculation unit
100 Skin
101 Perspiration
110, 111 Opening

1.-7. (canceled)

8. A device comprising:
a base member including a through-hole, a first end portion of the through-hole opening on a first side of the base member, a second end portion of the through-hole opening on a second side of the base member, the second side opposite the first side;
a first sensor element in the through-hole, the first sensor element configured to detect a signal related to an electrical characteristic of a liquid in the through-hole; and
a porous body on an inner wall of the through-hole and on a surface of the base member, the porous body disposed on the inner wall in a portion farther from a first position of the first sensor element when viewed from the first end portion of the through-hole, the surface of the base member being on the second side of the base member, the porous body having hydrophilicity.

9. The device of claim 8, wherein:
when the base member is attached to a body of a wearer with the base member facing a skin of the wearer, the first side of the base member faces the skin of the wearer,
the first sensor element is configured to detect an electrical signal derived from an analysis target component contained in perspiration that has flowed into the through-hole from the first end portion, and
at least the inner wall of the through-hole has hydrophilicity.

10. The device of claim 9, further comprising a water-repellent member provided on a surface of the base member on the first side of the base member.

11. The device of claim 9 further comprising:
a component concentration calculation circuit configured to calculate a value of a concentration of the analysis target component from the electrical signal detected by the first sensor element.

12. The device of claim 11, wherein the component concentration calculation circuit is further configured to determine acquisition of the concentration of the analysis target component is completed when the value of the concentration of the analysis target component is stable.

13. The device of claim 11 further comprising:
a second sensor element in the through-hole at a second position adjacent to the first sensor element,
wherein the component concentration calculation circuit is further configured to determine acquisition of the concentration of the analysis target component is completed when perspiration secreted from the skin of the wearer is detected by the second sensor element.

14. The device of claim 11, further comprising a communication circuit configured to transmit, to an external device, the value of the concentration of the analysis target component calculated by the component concentration calculation circuit.

15. A device comprising:
a base member having a through-hole extending from a first surface of the base member to a second surface of the base member, the second surface opposite the first surface, the through-hole having hydrophilicity;
a porous body having a first portion on the first surface of the base member and having a second portion in the through-hole, the porous body having hydrophilicity;
a first sensor element in the through-hole, the first sensor element disposed closer to the second surface of the base member than the second portion of the porous body, the first sensor element configured to detect an electrical signal derived from a target component contained in a liquid in the through-hole; and
a control circuit configured to calculate a concentration of the target component from the electrical signal detected by the first sensor element.

16. The device of claim 15 further comprising:
a water-repellent member on the second surface of the base member.

17. The device of claim 15 further comprising:
a second sensor element in the through-hole, the second sensor element disposed closer to the second surface of the base member than the second portion of the porous body, the second sensor element configured to detect the liquid in the through-hole has reached a position adjacent to the second sensor element; and
a communication circuit,
wherein the control circuit is further configured to control the communication circuit to transmit the concentration of the target component to an external device in response to the second sensor element detecting the liquid in the through-hole has reached the position adjacent to the second sensor element.

18. The device of claim 15, wherein the target component is lactic acid.

19. The device of claim 15, wherein the target component is glucose.

20. The device of claim 15, wherein the target component is a sodium ion.

21. The device of claim 15, wherein the target component is a potassium ion.

22. The device of claim 15, wherein the first sensor element comprises an ion selective electrode.

23. The device of claim 15, wherein the first sensor element comprises an enzyme electrode.

24. The device of claim 15, wherein the first sensor element comprises an ion-sensitive field effect transistor.

25. The device of claim 15, wherein the base member comprises nylon.

26. The device of claim 15, wherein the base member comprises cellulose.
